(19) United States
(12) Patent Application Publication
(10) Pub. No.: US 2025/0102333 A1
(43) Pub. Date: Mar. 27, 2025

(54) SENSOR TESTING STATION

(71) Applicant: AutoStore Technology AS, Nedre Vats (NO)

(72) Inventors: Ivar Fjeldheim, Haugesund (NO); Trond Austrheim, Etne (NO)

(21) Appl. No.: 18/912,155

(22) Filed: Oct. 10, 2024

Related U.S. Application Data
(63) Continuation of application No. 17/796,020, filed on Jul. 28, 2022, now Pat. No. 12,146,770.

(30) Foreign Application Priority Data
Jan. 31, 2020 (NO) 20200120

(51) Int. Cl. G01D 21/02; A61L 2/10; A61L 2/24; B65G 1/04

(52) U.S. Cl. CPC G01D 21/02; A61L 2/10; A61L 2/24; B65G 1/0464; A61L 2202/11; A61L 2202/14

(57) ABSTRACT

A system performs measurements in storage containers for storing items. The storage containers are stored in an automated storage system including a framework structure forming a three-dimensional storage grid structure for storing the storage containers. The grid structure forms vertical storage columns each having a horizontal area defined by the size of an access opening of the vertical storage columns. A rail system is arranged on the framework structure defining the circumference of each access opening on top of each storage column. The rail system provides available routes for container handling vehicles handling and transferring the storage containers to and from the storage columns. The system further includes a testing station, accessible to a container handling vehicle via the rail system, with measuring equipment for measuring atmospheric conditions and for performing measurements in said storage container. The testing station is configured to communicate measurement data to a computer system. The testing station includes an upper part to which a measuring platform with measuring equipment is attached, a lower part for holding a container, and connector for connecting the upper part and the lower part and the testing station includes a lifting device adapted to raise and lower the measuring platform.

### BENEFIT CLAIM

This application claims the benefit under 35 U.S.C. § 120 as a continuation of application Ser. No. 17/796,020, filed 28 Jul. 2022, which is a national stage entry of PCT international application PCT/EP2021/051779, filed 26 Jan. 2021, which claims priority to Norwegian Patent Application No. 20200120, filed on Jan. 31, 2020, the entire contents of which are hereby incorporated by reference for all purposes as if fully set forth herein. The applicant rescinds any disclaimer of subject matter in any priority application and advises the USPTO that the claims of this application may be broader than the claims of any priority application.

### FIELD OF THE INVENTION

The present invention relates to an automated storage and retrieval system for storage and retrieval of containers, in particular to a system and method for performing measurements in a container when placed in a testing station comprising measuring equipment.

### BACKGROUND AND PRIOR ART

FIG. 1 discloses a typical prior art automated storage and retrieval system 1 with a framework structure 100 and FIGS. 2 and 3 discloses two different prior art container handling vehicles 201,301 suitable for operating on such a system 1.

The framework structure 100 comprises upright members 102, horizontal members 103 and a storage volume comprising storage columns 105 arranged in rows between the upright members 102 and the horizontal members 103. In these storage columns 105 storage containers 106, also known as bins, are stacked one on top of one another to form stacks 107. The members 102, 103 may typically be made of metal, e.g. extruded aluminum profiles.

The framework structure 100 of the automated storage and retrieval system 1 comprises a rail system 108 arranged across the top of framework structure 100, on which rail system 108 a plurality of container handling vehicles 201,301 are operated to raise storage containers 106 from, and lower storage containers 106 into, the storage columns 105, and also to transport the storage containers 106 above the storage columns 105. The rail system 108 comprises a first set of parallel rails 110 arranged to guide movement of the container handling vehicles 201,301 in a first direction X across the top of the frame structure 100, and a second set of parallel rails 111 arranged perpendicular to the first set of rails 110 to guide movement of the container handling vehicles 201,301 in a second direction Y which is perpendicular to the first direction X. Containers 106 stored in the columns 105 are accessed by the container handling vehicles through access openings 112 in the rail system 108. The container handling vehicles 201,301 can move laterally above the storage columns 105, i.e. in a plane which is parallel to the horizontal X-Y plane.

The upright members 102 of the framework structure 100 may be used to guide the storage containers during raising of the containers out from and lowering of the containers into the columns 105. The stacks 107 of containers 106 are typically self-supportive.

Each prior art container handling vehicle 201,301 comprises a vehicle body 201a,301a, and first and second sets of wheels 201b,301b,201c,301c which enable the lateral movement of the container handling vehicles 201,301 in the X direction and in the Y direction, respectively. In FIGS. 2 and 3 two wheels in each set are fully visible. The first set of wheels 201b,301b is arranged to engage with two adjacent rails of the first set 110 of rails, and the second set of wheels 201c,301c is arranged to engage with two adjacent rails of the second set 111 of rails. At least one of set wheels 201b,301b,201c,301c can be lifted and lowered, so that the first set of wheels 201b,301b and/or the second set of wheels 201c,301c can be engaged with the respective set of rails 110, 111 at any one time.

Each prior art container handling vehicle 201,301 also comprises a lifting device (not shown) for vertical transportation of storage containers 106, e.g. raising a storage container 106 from, and lowering a storage container 106 into, a storage column 105. The lifting device comprises one or more gripping/engaging devices which are adapted to engage a storage container 106, and which gripping/engaging devices can be lowered from the vehicle 201,301 so that the position of the gripping/engaging devices with respect to the vehicle 201,301 can be adjusted in a third direction Z which is orthogonal the first direction X and the second direction Y. Parts of the gripping device of the container handling vehicle 301 is shown in in FIG. 3 and is indicated with reference number 304. The gripping device of the container handling device 201 is located within the vehicle body 301a in FIG. 2.

Conventionally, and also for the purpose of this application, Z=1 identifies the uppermost layer of storage containers, i.e. the layer immediately below the rail system 108, Z=2 the second layer below the rail system 108, Z=3 the third layer etc. In the exemplary prior art disclosed in FIG. 1, Z=8 identifies the lowermost, bottom layer of storage containers. Similarly, X=1 . . . n and Y=1 . . . n identifies the position of each storage column 105 in the horizontal plane. Consequently, as an example, and using the Cartesian coordinate system X, Y, Z indicated in FIG. 1, the storage container identified as 106′ in FIG. 1 can be said to occupy storage position X=10, Y=2, Z=3. The container handling vehicles 201,301 can be said to travel in layer Z=0, and each storage column 105 can be identified by its X and Y coordinates.

The storage volume of the framework structure 100 has often been referred to as a grid 104, where the possible storage positions within this grid is referred to as a storage cell. Each storage column may be identified by a position in an X- and Y-direction, while each storage cell may be identified by a container number in the X-, Y and Z-direction.

Each prior art container handling vehicle 201,301 comprises a storage compartment or space for receiving and stowing a storage container 106 when transporting the storage container 106 across the rail system 108. The storage space may comprise a cavity arranged centrally within the vehicle body 201a as shown in FIG. 2 and as described in e.g. WO2015/193278A1, the contents of which are incorporated herein by reference.

FIG. 3 shows an alternative configuration of a container handling vehicle 301 with a cantilever construction. Such a vehicle is described in detail in e.g. NO317366, the contents of which are also incorporated herein by reference.

The central cavity container handling vehicles 201 shown in FIG. 2 may have a footprint that covers an area with dimensions in the X and Y directions which is generally equal to the lateral extent of a storage column 105, e.g. as is described in WO2015/193278A1, the contents of which are incorporated herein by reference. The term ‘lateral’ used herein may mean ‘horizontal’.

Alternatively, the central cavity container handling vehicles 101 may have a footprint which is larger than the lateral area defined by a storage column 105, e.g. as is disclosed in WO2014/090684A1.

The rail system 108 typically comprises rails with grooves into which the wheels of the vehicles are inserted. Alternatively, the rails may comprise upwardly protruding elements, where the wheels of the vehicles comprise flanges to prevent derailing. These grooves and upwardly protruding elements are collectively known as tracks. Each rail may comprise one track, or each rail may comprise two parallel tracks.

WO2018146304, the contents of which are incorporated herein by reference, illustrates a typical configuration of rail system 108 comprising rails and parallel tracks in both X and Y directions.

In the framework structure 100, a majority of the columns 105 are storage columns 105, i.e. columns 105 where storage containers 106 are stored in stacks 107. However, some columns 105 may have other purposes. In FIG. 1, columns 119 and 120 are such special-purpose columns used by the container handling vehicles 201,301 to drop off and/or pick up storage containers 106 so that they can be transported to an access station (not shown) where the storage containers 106 can be accessed from outside of the framework structure 100 or transferred out of or into the framework structure 100. Within the art, such a location is normally referred to as a ‘port’ and the column in which the port is located may be referred to as a ‘port column’ 119,120. The transportation to the access station may be in any direction, that is horizontal, tilted and/or vertical. For example, the storage containers 106 may be placed in a random or dedicated column 105 within the framework structure 100, then picked up by any container handling vehicle and transported to a port column 119,120 for further transportation to an access station. Note that the term ‘tilted’ means transportation of storage containers 106 having a general transportation orientation somewhere between horizontal and vertical.

In FIG. 1, the first port column 119 may for example be a dedicated drop-off port column where the container handling vehicles 201,301 can drop off storage containers 106 to be transported to an access or a transfer station, and the second port column 120 may be a dedicated pick-up port column where the container handling vehicles 201,301 can pick up storage containers 106 that have been transported from an access or a transfer station.

The access station may typically be a picking or a stocking station where product items are removed from or positioned into the storage containers 106. In a picking or a stocking station, the storage containers 106 are normally not removed from the automated storage and retrieval system 1, but are returned into the framework structure 100 again once accessed. A port can also be used for transferring storage containers to another storage facility (e.g. to another framework structure or to another automated storage and retrieval system), to a transport vehicle (e.g. a train or a lorry), or to a production facility.

A conveyor system comprising conveyors is normally employed to transport the storage containers between the port columns 119,120 and the access station.

If the port columns 119,120 and the access station are located at different levels, the conveyor system may comprise a lift device with a vertical component for transporting the storage containers 106 vertically between the port column 119,120 and the access station.

The conveyor system may be arranged to transfer storage containers 106 between different framework structures, e.g. as is described in WO2014/075937A1, the contents of which are incorporated herein by reference.

When a storage container 106 stored in one of the columns 105 disclosed in FIG. 1 is to be accessed, one of the container handling vehicles 201,301 is instructed to retrieve the target storage container 106 from its position and transport it to the drop-off port column 119. This operation involves moving the container handling vehicle 201,301 to a location above the storage column 105 in which the target storage container 106 is positioned, retrieving the storage container 106 from the storage column 105 using the container handling vehicle's 201,301 lifting device (not shown), and transporting the storage container 106 to the drop-off port column 119. If the target storage container 106 is located deep within a stack 107, i.e. with one or a plurality of other storage containers 106 positioned above the target storage container 106, the operation also involves temporarily moving the above-positioned storage containers prior to lifting the target storage container 106 from the storage column 105. This step, which is sometimes referred to as “digging” within the art, may be performed with the same container handling vehicle that is subsequently used for transporting the target storage container to the drop-off port column 119, or with one or a plurality of other cooperating container handling vehicles. Alternatively, or in addition, the automated storage and retrieval system 1 may have container handling vehicles specifically dedicated to the task of temporarily removing storage containers from a storage column 105. Once the target storage container 106 has been removed from the storage column 105, the temporarily removed storage containers can be repositioned into the original storage column 105. However, the removed storage containers may alternatively be relocated to other storage columns.

When a storage container 106 is to be stored in one of the columns 105, one of the container handling vehicles 201,301 is instructed to pick up the storage container 106 from the pick-up port column 120 and transport it to a location above the storage column 105 where it is to be stored. After any storage containers positioned at or above the target position within the storage column stack 107 have been removed, the container handling vehicle 201,301 positions the storage container 106 at the desired position. The removed storage containers may then be lowered back into the storage column 105, or relocated to other storage columns.

For monitoring and controlling the automated storage and retrieval system 1, e.g. monitoring and controlling the location of respective storage containers 106 within the framework structure 100, the content of each storage container 106; and the movement of the container handling vehicles 201,301 so that a desired storage container 106 can be delivered to the desired location at the desired time without the container handling vehicles 201,301 colliding with each other, the automated storage and retrieval system 1 comprises a control system 500 which typically is computerized and which typically comprises a database for keeping track of the storage containers 106.

FIG. 4 describes a delivery vehicle. The delivery vehicles comprise a base with the same setup of wheels as on the container handling vehicles. The wheel base unit features a wheel arrangement having a first set of wheels for movement in a first direction upon a rail grid (i.e. any of the top rail grid and the transfer rail grid) and a second set of wheels for movement in a second direction perpendicular to the first direction. Each set of wheels comprises two pairs of wheels arranged on opposite sides of the wheel base unit. To change the direction in which the wheel base unit may travel upon the rail grid, one of the sets of wheels is connected to a wheel displacement assembly. The wheel displacement assembly is able to lift and lower the connected set of wheels relative to the other set of wheels such that only the set of wheels travelling in a desired direction is in contact with the rail grid. The wheel displacement assembly is driven by an electric motor. Further, two electric motors, powered by a rechargeable battery, are connected to the set of wheels to move the wheel base unit in the desired direction. The horizontal periphery of the wheel base unit is dimensioned to fit within the horizontal area defined by a grid cell of the rail grid such that two-wheel base units may pass each other on any adjacent grid cells of the rail grid. In other words, the wheel base unit may have a footprint, i.e. an extent in the X and Y directions, which is generally equal to the horizontal area of a grid cell, i.e. the extent of a grid cell in the X and Y directions, e.g. as is described in WO2015/193278A1, the contents of which are incorporated herein by reference.

A problem with the prior art solutions is that if there are perishable items in the storage there is a need for measuring the freshness of the products and the conditions the items are stored in. There is however a problem with getting accurate readings of the items without having to turn to costly solutions that require expensive equipment or extensive rebuilds. It is therefore an object of the present invention to solve the problems mentioned above.

### SUMMARY OF THE INVENTION

The present invention is set forth and characterized in the independent claims, while the dependent claims describe other characteristics of the invention.

In one aspect, the invention is related to a system for performing measurements in storage containers for storing items, the storage containers are stored in an automated storage system comprising a framework structure forming a three-dimensional storage grid structure for storing the storage containers, where the grid structure forms vertical storage columns each having a horizontal area defined by the size of an access opening of the vertical storage columns and where a rail system is arranged on the framework structure defining the circumference of each access opening on top of each storage column, the rail system providing available routes for container handling vehicles handling and transferring the storage containers to and from the storage columns, and wherein said system is wherein the system further comprises a testing station, accessible to a container handling vehicle via the rail system, with measuring equipment for measuring atmospheric conditions and for performing measurements in said storage container and where the testing station is configured to communicate measurement data to a computer system

Further the testing station may comprise an upper part to which the measuring platform is attached, a lower part for receiving the container and a part connecting the upper and the lower part. Also the testing station may have a space for accommodating a container between the upper and the lower part, and the testing station may be adapted to raise and lower the measuring platform.

The testing station may be adapted to accommodate a container handling vehicle with a container on top.

The measuring platform may be raised and lowered by a rope, band or wire controlled by an electric motor, or the measuring platform may be raised and lowered by a robotic arm, and the measuring platform comprises temperature measurement equipment, moisture detectors, gas detectors and cameras.

In a third aspect the invention is directed to a method of performing measurements in a container for storing in an automated storage system comprising a three-dimensional grid (4) of an underlying storage system (1), at least one container handling vehicle for transporting containers, ports for picking items for further distribution, a central computer system for controlling the operation of the storage system, and wherein said method comprises the steps of:
placing a container in a testing station by means of a container handling vehicle,
lowering a measuring platform attached to the testing station onto the container,
performing measurements, transmitting the measurements to the central computer system, performing an analysis based upon the measurements, transmitting instructions based on the analysis to a container handling vehicle, raising the measuring platform, transporting the container to another destination by means of a container handling vehicle. Performing measurements of temperature, moisture and gas level in the container and by using cameras for visual inspection of the items.

### BRIEF DESCRIPTION OF THE DRAWINGS

Following drawings are appended to facilitate the understanding of the invention. The drawings show embodiments of the invention, which will now be described by way of example only, where:

FIG. 1 is a perspective view of a framework structure of a prior art automated storage and retrieval system.

FIG. 2 is a perspective view of a prior art container handling vehicle having a centrally arranged cavity for carrying storage containers therein.

FIG. 3 is a perspective view of a prior art container handling vehicle having a cantilever for carrying storage containers underneath.

FIG. 4 is a side view of a delivery vehicle.

FIG. 5A, FIG. 5B, FIG. 5C, and FIG. 5D is a side view of an embodiment of the present invention wherein a container handling vehicle with a central cavity places a container in a testing station for measurements.

FIG. 6A, FIG. 6B, FIG. 6C, and FIG. 6D is a side view of an embodiment of the present invention wherein a container handling vehicle with a cantilever solution places a container in a testing station for measurements.

FIG. 7A, FIG. 7B, and FIG. 7C is a side view of an embodiment of the present invention wherein a delivery vehicle carrying a container is placed in a testing station.

FIG. 8A, FIG. 8B, FIG. 8C, and FIG. 8D is a side view of an alternative embodiment of the present invention wherein a container handling vehicle with a cantilever solution places a container in a testing station for measurements.

FIG. 9A, FIG. 9B, and FIG. 9C is a side view of an alternative embodiment of the present invention wherein a delivery vehicle carrying a container is placed in a testing station.

FIG. 10 is a flowchart describing the steps in the process of the embodiments of the present invention.

### DETAILED DESCRIPTION OF THE INVENTION

In the following, embodiments of the invention will be discussed in more detail with reference to the appended drawings. It should be understood, however, that the drawings are not intended to limit the invention to the subject-matter depicted in the drawings.

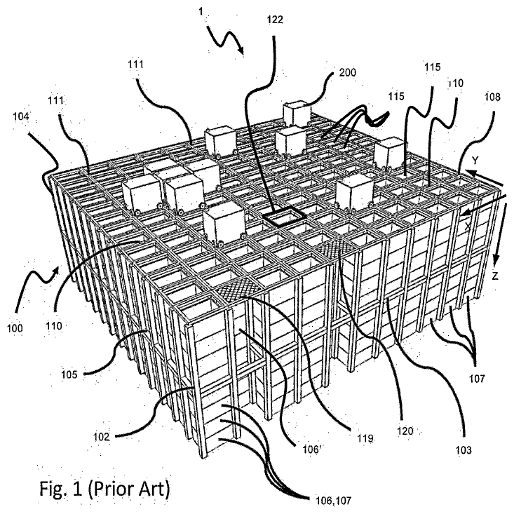

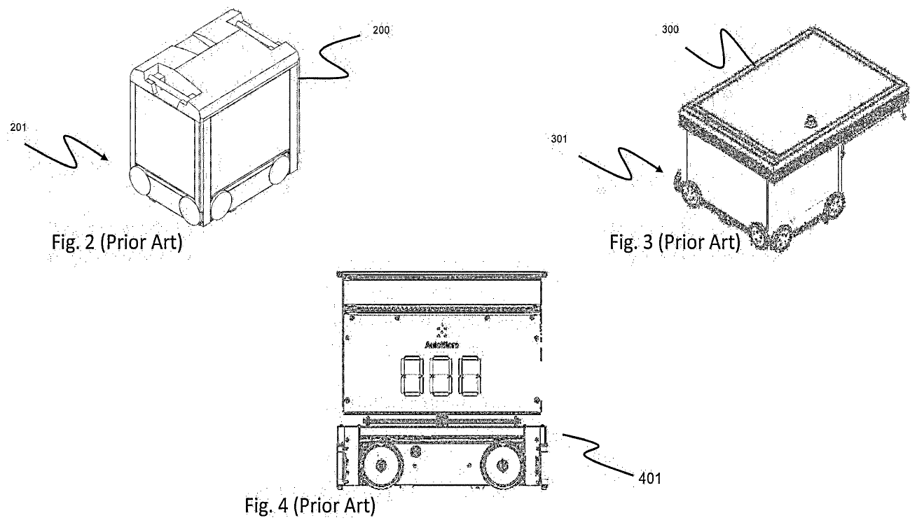

The framework structure 100 of the automated storage and retrieval system 1 is constructed in accordance with the prior art framework structure 100 described above in connection with FIGS. 1-3, i.e. a number of upright members 102 and a number of horizontal members 103, which are supported by the upright members 102, and further that the framework structure 100 comprises a first, upper rail system 108 in the X direction and Y direction.

The framework structure 100 further comprises storage compartments in the form of storage columns 105 provided between the upright members 102, 103, where storage containers 106 are stackable in stacks 107 within the storage columns 105.

The framework structure 100 can be of any size. In particular it is understood that the framework structure can be considerably wider and/or longer and/or deeper than disclosed in FIG. 1. For example, the framework structure 100 may have a horizontal extent of more than 700×700 columns and a storage depth of more than twelve containers.

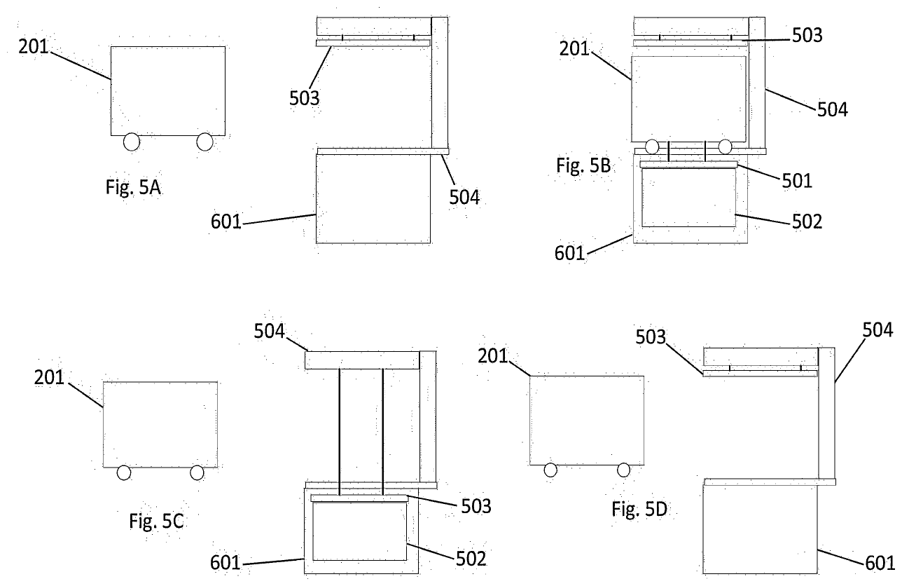

Embodiments of the automated storage and retrieval system according to the invention will now be discussed in more detail with reference to FIGS. 5A-D, 6A-D, 7A-C, 8A-D, 9A-C and 10.

In a preferred embodiment of the present invention the testing station is comprised of a top portion. This top portion houses the measuring platform. The measuring platform comprises at least one sensor. The at least one sensor is used to measure a at least one atmospheric condition in a storage container. One such atmospheric condition might be the temperature in the container, further the sensor can be used to detect the presence of gasses that is released during rotting of food. One such gas can be methane. Other gasses that are released during decay of food is carbon dioxide and hydrogen sulphide. In the case of carbon dioxide and hydrogen sulphide they are heavier than air and will therefore collect at the bottom of the container. The sensors for sensing carbon dioxide and hydrogen sulphide can therefore be attached to a probe that is lowered into the container. Alternatively there can be holes in the sides or in the bottom of the containers into which it is possible to insert sensors. In yet another solution there can be a slit or a tunnel e.g. along the inside edges of the container wherein the probe can be lowered into the bottom of the container without the content of the container hindering the probe. Further there can be detectors to detect moisture. The presense of moisture is due to the fact that during rotting of food the cells of the food is broken down and the liquid in the cells leaks out. A further type of measuring equipment can be a camera. The camera can take pictures into the container, i.e., of the contents, in order to detect if there is any sign of rotting food. The camera can be an ordinary camera that takes colour images in order to detect if there is marks on the food, like e.g., brown spots on bananas, or discoloration on the surface due to milldue. Alternatively or in addition, there can be used a camera that has ultraviolet light in order to detect mold.

There is also the possibility to use UV light in order to kill bacteria on the food. There is further a possibility to use UV light to kill mold. Therefor the measuring platform can have a UV light source that can be used to both detect rotting food and to kill bacteria and mold on the food and in the container. The difference between using the UV light source to detect decay and to kill bacteria and mold is the wavelength of the light and the power of the light souce.

FIG. 5A, FIG. 5B, FIG. 5C, and FIG. 5D is a side view of an embodiment of the present invention wherein a container handling vehicle with a central cavity places a container in a testing station for measurements. The container handling vehicle with a container in its central cavity moves into the testing station. The container is lowered down into a cavity underneath the container handling vehicle. After the container handling vehicle has placed the container in the testing station, the container handling vehicle moves away. After it has moved away, the measuring equipment is lowered down on a measuring platform. The testing station lowers a measuring platform onto the container. The measuring platform is lowered using lines attached to an electric motor. These lines can be wire, belts, chains, rope or similar. The measuring platform fits over the top opening. The platform further comprises measuring equipment attached to the underside. The measuring equipment is placed so that it fits inside the container when the platform is placed on the container. The measuring equipment can be a temperature sensor, a moisture sensor, a gas sensor and/or cameras. Any other kind of measuring equipment that can be mounted onto the measuring platform can be used. After the measuring equipment has collected the data, it can be sent to a central computer system. The central computer system can store the data together with the ID of the container in order to keep track of the condition of the content of the container. Based on the conditions of the content of the container, the central computer system can send instruction to a container handling vehicle to transport the container to a destination dependent on the condition of the items in the container. If the items are in a contidition that is acceptable for distribution to customers, the container with the items are transported either to a station where the desired items can be picked for further distribution, or the container with the items can be transported back into the storage system. If one or more of the items in the container is of poor quality the container can be transported to a destination where the spoiled items can be removed from the container. After the items have been removed the container is either transported back to the storage system or to a port for further distribution to a picking station where items are picked for further distribution to customers.

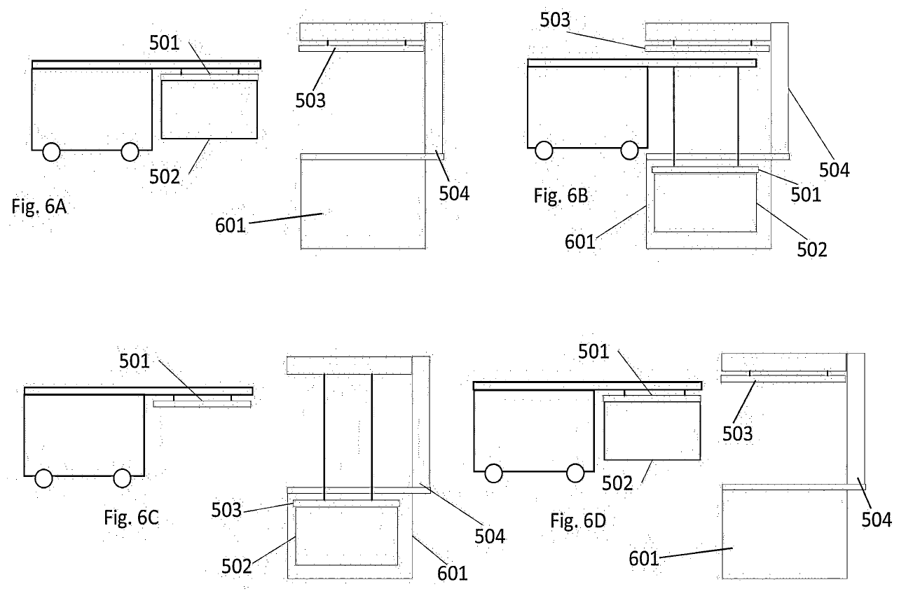

FIG. 6A, FIG. 6B, FIG. 6C, and FIG. 6D is a side view of an embodiment of the present invention wherein a container handling vehicle with a cantilever solution places a container in a testing station for measurements. The container handling vehicle approaches the testing station carrying a container from the storage system. The container handling vehicle places the container in the testing station. The container handling vehicle backs away giving the testing station access to the contents of the container via its top opening. The testing station lowers a measuring platform onto the container. The measuring platform is lowered using lines attached to an electric motor. These lines can be wire, belts, chains, rope or similar. The measuring platform fits over the top opening. The platform comprises measuring equipment attached to the underside. The measuring equipment is placed, for example, on the underside, so that it fits inside the container when the platform is placed on the container. The measuring equipment can be a temperature sensor, a moisture sensor, a gas sensor and/or cameras. Any other kind of measuring equipment that can be mounted onto the measuring platform can be used. The collected data can be sent to a central computer system. The central computer system can store and analyse the data. The data can be stored together with the ID of the container in order to keep track of the condition of the content of the container. Based on the conditions of the content of the container, the central computer system can send instruction to a container handling vehicle to transport the container to a destination dependent on the condition of the items in the container. If the items are in a contidition that is acceptable for distribution to customers, the container with the items are transported either to a station where the desired items can be picked for further distribution, or the container with the items can be transported back into the storage system. If one or more of the items in the container is of poor quality the container can be transported to a destination where the spoiled items can be removed from the container. After the items have been removed the container is either transported back to the storage system or to a port for further distribution to a picking station where items are picked for further distribution to customers.

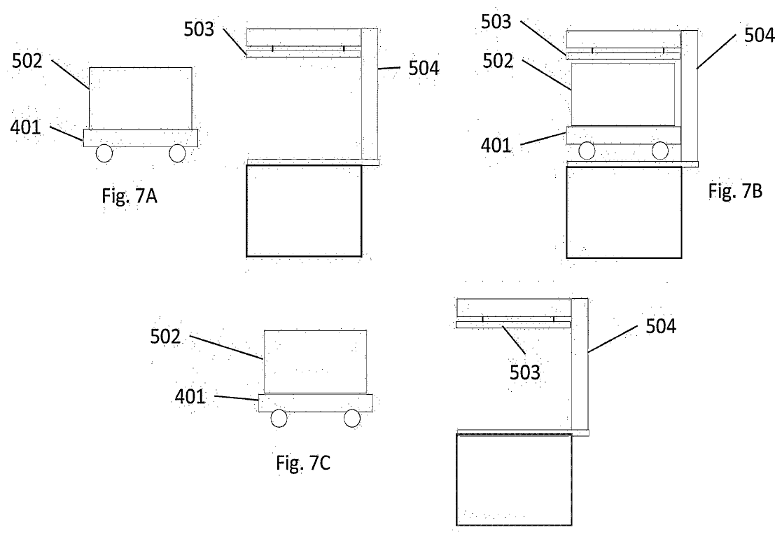

FIG. 7A, FIG. 7B, and FIG. 7C is a side view of an embodiment of the present invention wherein a delivery vehicle carrying a container is placed in a testing station. Since the delivery vehicle does not have a lifting platform arranged above the container, like the previously described handling vehicles, it is not possible for the delivery vehicle to place the container in the testing station with out help. In an embodiment of the present invention the delivery vehicle with the container on top therefor fits into the testing station. In this embodiment the testing station therefor has tracks in the bottom in order to allow the delivery vehicle to manouever itself with the container on top into the testing station. The testing station lowers the measuring platform onto the container. The measuring platform is lowered using, wire, belts, chains, rope or similar attached to an electric motor. The measuring platform fits over the top opening. The platform further comprises measuring equipment attached to the underside. The measuring equipment is placed so that it fits inside the container when the platform is placed on the container. The measuring equipment can be a temperature sensor, a moisture sensor, a gas sensor and/or cameras. Any other kind of measuring equipment that can be mounted onto the measuring platform can be used. After the data from the measuring equipment has been collected the data can be sent over to the central computer system. When the data is collected the delivery vehicle with the container on top can be transported to the next destination.

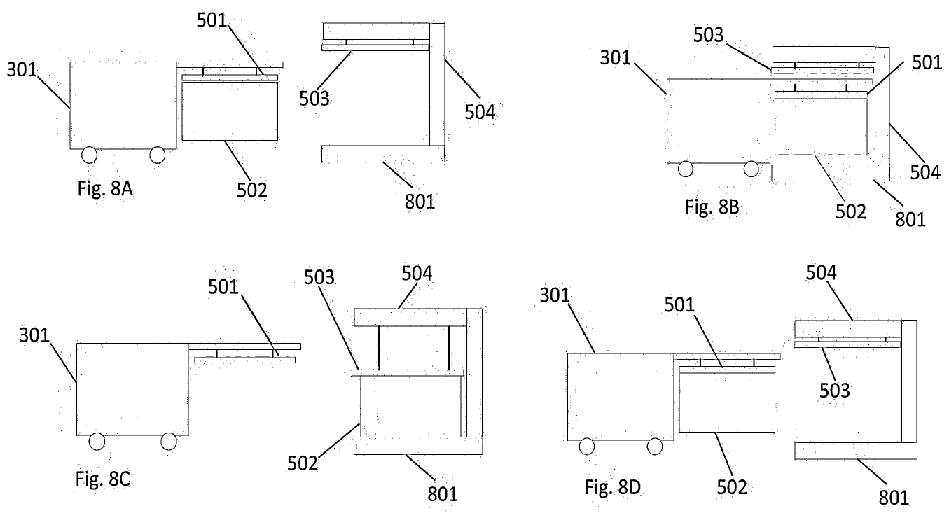

FIG. 8A, FIG. 8B, FIG. 8C, and FIG. 8D is a side view of an alternative embodiment of the present invention wherein a container handling vehicle with a cantilever solution places a container in a testing station for measurements. The container handling vehicle approaches the testing station carrying a container from the storage system. The container handling vehicle places the container on the base of the testing station. The container handling vehicle backs away giving the testing station access to the contents of the container via its top opening. The testing station lowers a measuring platform onto the container. The measuring platform is lowered using, wire, belts, chains, rope or similar attached to an electric motor. The measuring platform fits over the top opening. The platform further comprises measuring equipment attached to the underside. The measuring equipment are placed so that it fits inside the container when the platform is placed on the container. The measuring equipment can be a temperature sensor, a moisture sensor, a gas sensor and/or cameras. Any other kind of measuring equipment that can be mounted onto the measuring platform can be used. The collected data can be sent to a central computer system. The central computer system can store and analyse the data. The data can be stored together with the ID of the container in order to keep track of the condition of the content of the container. Based on the conditions of the content of the container, the central computer system can send instruction to a container handling vehicle to transport the container to a destination dependent on the condition of the items in the container. If the items are in a condition that is acceptable for distribution to customers, the container with the items are transported either to a station where the desired items can be picked for further distribution, or the container with the items can be transported back into the storage system. If one or more of the items in the container is of poor quality the container can be transported to a destination where the spoiled items can be removed from the container. After the items have been removed the container is either transported back to the storage system or to a port for further distribution to a picking station where items are picked for further distribution to customers.

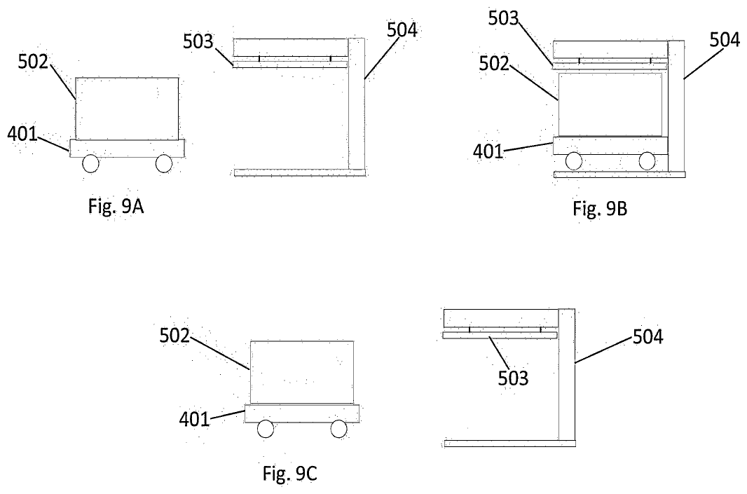

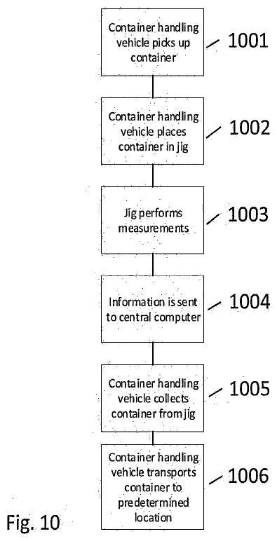

FIG. 9A, FIG. 9B, and FIG. 9C is a side view of an alternative embodiment of the present invention wherein a delivery vehicle carrying a container is placed in a testing station. Since the delivery vehicle does not have a lifting platform holding the container it is not possible for the delivery vehicle to place the container in the testing station with out help. In an embodiment of the present invention the delivery vehicle with the container on top therefor fits into the testing station. In this embodiment the testing station has tracks in the base of the testing station in order to allow the delivery vehicle to manouever itself with the container on top into the testing station. The testing station lowers the measuring platform onto the container. The measuring platform is lowered using, wire, belts, chains, rope or similar attached to an electric motor. The measuring platform fits over the top opening. The platform further comprises measuring equipment attached to the underside. The measuring equipment are placed so that it fits inside the container when the platform is placed on the container. The measuring equipment can be a temperature sensor, a moisture sensor, a gas sensor and/or cameras. Any other kind of measuring equipment that can be mounted onto the measuring platform can be used. After the data from the measuring equipment has been collected the data can be sent over to the central computer system. When the data is collected the delivery vehicle with the container on top can be transported to the next destination.

FIG. 10 is a flowchart describing the steps in the process of the embodiments of the present invention. a container handling vehicle or a delivery vehicle transports a container from the storage system. The storage system is an automated storage and retrieval system 1 with a framework structure 100, comprising upright members 102, horizontal members 103 and a storage volume comprising storage columns 105 arranged in rows between the upright members 102 and the horizontal members 103. In these storage columns 105 storage containers 106, also known as bins, are stacked one on top of one another to form stacks 107. The container handling vehicle or the delivery vehicle transports the container to the measuring station. Dependent on the vehicle transporting the container, the container is either placed in the testing station or the delivery vehicle with the container on top is parked in the testing station. The testing station lowers the measuring platform, the measuring platform fits over the top of the container. The measuring platform has sensors attached to it. The sensors can be a temperature sensor, a gas sensor, a moisture detector and/or a camera. The data from the sensors are collected and sent to the central computer system. In the central computer system the collected data is stored together with the ID of the container. Further the data can be analyzed and the result to the analysis is the basis for the conclution of where the container and its content go after the measuring. After the measuring is performed, the central computer system sends instructions to the container handling vehicle or the delivery vehicle telling it to transport the container to a predetermined destination. The predetermined destination is dependent on the quality of the items in the container. If the items are of a quality that is good enough to be distributed to consumers, the container is either transported to a port and from there it is transported to a picking station where items are collected for further distribution, alternatively the container can be transported back to the storage grid structure. If the items in the container is of a quality that is not good enough for human consumption, it is transported to a destination where the spoiled items are picked from the container and disposed of according to prescribed instructions.

In the preceding description, various aspects of the delivery vehicle and the automated storage and retrieval system according to the invention have been described with reference to the illustrative embodiment. For purposes of explanation, specific numbers, systems and configurations were set forth in order to provide a thorough understanding of the system and its workings. However, this description is not intended to be construed in a limiting sense.

Various modifications and variations of the illustrative embodiment, as well as other embodiments of the system, which are apparent to persons skilled in the art to which the disclosed subject matter pertains, are deemed to lie within the scope of the present invention.

### LIST OF REFERENCE NUMBERS

### Prior art (FIGS. 1-10):

100 Framework structure
102 Upright members of framework structure
103 Horizontal members of framework structure
104 Storage grid
105 Storage column
106 Storage container
106′ Particular position of storage container
107 Stack
108 Rail system
110 Parallel rails in first direction (X)
110a First rail in first direction (X)
110b Second rail in first direction (X)
111 Parallel rail in second direction (Y)
111a First rail of second direction (Y)
111b Second rail of second direction (Y)
112 Access opening
119 First port column
120 Second port column
201 Prior art storage container vehicle
201a Vehicle body of the storage container vehicle 101
201b Drive means/wheel arrangement, first direction (X)
201c Drive means/wheel arrangement, second direction (Y)
301 Prior art cantilever storage container vehicle
301a Vehicle body of the storage container vehicle 101
301b Drive means in first direction (X)
301c Drive means in second direction (Y)
X First direction
Y Second direction
Z Third direction
401 Delivery vehicle
501 Container lifting platform
502 Container
503 Measuring platform
504 Testing station
601 Container placement area.
801 Plate

What is claimed is:

1. A system for performing measurements in storage containers for storing items, wherein the storage containers are configured to be stored in an automated storage system, the automated storage system comprising:
a framework structure forming a three-dimensional storage grid structure for storing the storage containers in storage columns;
a first vehicle for carrying a storage container; and
a rail system arranged on the framework structure providing available routes for vehicles handling and transferring the storage containers to and from the storage columns;
wherein the system for performing measurements comprises a testing station comprising measuring equipment for measuring conditions in the storage container, wherein the testing station is configured to communicate measurement data to a computer system, wherein the testing station is accessible to the first vehicle via the rail system.

2. The system of claim 1, wherein the testing station is configured to position the measuring equipment onto the storage container.

3. The system of claim 1, wherein the system is configured to raise or lower the measuring equipment.

4. The system of claim 3, wherein the system is configured to raise or lower the measuring equipment by:
a rope, band, or wire controlled by an electric motor; or
a robotic arm.

5. The system of claim 1, wherein the measuring equipment comprises a sensor, wherein the storage container comprises a side having a hole for inserting the sensor into.

6. The system of claim 1, wherein the measuring equipment comprises a sensor, wherein the storage container comprises a slit or tunnel along an inside edge of the storage container for lowering the sensor into a bottom of the storage container.

7. The system of claim 1, wherein the measuring equipment comprises:
temperature measurement equipment;
a moisture detector;
a gas detector; and/or
a camera for visual inspection of items stored in the storage container.

8. The system of claim 1, wherein the conditions comprise a condition of contents of the storage container.

9. The system of claim 1, wherein the testing station comprises a measuring platform configured to be lowered onto the storage container, the measuring equipment being mounted onto the measuring platform, wherein the measuring platform is configured to fit over a top opening of the storage container.

10. The system of claim 9, wherein the measuring equipment is on the measuring platform and inside the storage container when the measuring platform is on the storage container.

11. A method for performing measurements in storage containers for storing items using a testing station, wherein the storage containers are configured to be stored in an automated storage system, the automated storage system comprising:
a framework structure forming a three-dimensional storage grid structure for storing the storage containers in storage columns;
a first vehicle for carrying a storage container; and
a rail system arranged on the framework structure providing available routes for vehicles handling and transferring the storage containers to and from the storage columns, wherein each of the vehicles comprises a vehicle controller communicating with a central computer system controlling operation of the automated storage system;
wherein the method comprises:
placing a storage container in the testing station by the first vehicle;
positioning the storage container onto measuring equipment attached to the testing station for measuring conditions in the storage container;
performing measurements to collect measurement data; and
transmitting the measurement data to a computer system.

12. The method of claim 11, wherein the method comprises:
raising the measuring equipment; and
transporting the storage container to a next destination by the first vehicle.

13. The method of claim 11, wherein the method comprises:
after the transmitting, storing the measurement data and performing an analysis of the measurement data; and
transmitting instructions, regarding where to transport the storage container, from the central computer system to the first vehicle based on a result of the analysis.

14. The method of claim 11, further comprising performing measurements by performing measurements of temperature, moisture and/or gas level in the storage container and/or by using cameras for visual inspection of the items.

15. The method of claim 11, further comprising raising or lowering the measuring equipment by a rope, band, or wire controlled by an electric motor.

16. The method of claim 11, further comprising raising or lowering the measuring equipment by a robotic arm.

17. The method of claim 11, wherein the measuring equipment comprises a sensor, wherein the method comprises inserting the sensor into a hole in a side of the storage container.

18. The method of claim 11, wherein the measuring equipment comprises a sensor, wherein the method comprises lowering the sensor into a bottom of the storage container using a slit or tunnel along an inside edge of the storage container.

19. The method of claim 11, wherein the measuring equipment is on a measuring platform, wherein the method further comprises placing the measuring platform on the storage container with the measuring equipment inside the storage container.
